(19) United States
(12) Patent Application Publication
(10) Pub. No.: US 2026/0263684 A1
(43) Pub. Date: Sep. 10, 2026

(54) PORTABLE PATIENT CARE KIT

(71) Applicant: SANOFI, Paris (FR)

(72) Inventor: Michael HELMER, Frankfurt am Main (DE)

(21) Appl. No.: 19/167,528

(22) PCT Filed: Mar. 20, 2024
(86) PCT No.: PCT/EP2024/057360

§ 371 Date: Sep. 22, 2025

(30) Foreign Application Priority Data
Mar. 23, 2023 (EP) 23315059.8

(51) Int. Cl. A61M 5/00

(52) U.S. Cl. CPC A61M 5/002; A61M 5/008

(57) ABSTRACT

A portable patient care kit is provided. The kit includes a housing with first and second housing components, the second component being movably connected to the first to reconfigure the housing between a closed state and a use state. At least one compartment is located on an interior surface of the first or second housing component and is configured to receive an item selected from a medical device, a medical device accessory, a medicament container, dressing material, a portable electronic device, an illumination device, or an optical device. A holder with a mount is provided to fasten the item or an external item to the holder, the holder being connected or connectable to at least one housing component to protrude from the interior when the housing is in the use state.

### CROSS-REFERENCE TO RELATED APPLICATIONS

The present application is the national stage entry of International Patent Application No. PCT/EP 2024/057360, filed on Mar. 20, 2024 and claims priority to European Application No. EP 23315059.8, filed on Mar. 23, 2023, the disclosures of which are incorporated herein by reference.

### TECHNICAL FIELD

The present disclosure relates to the field of patient care and in particular to a portable patient care kit as well as to a method of assisting a user in using medical devices, medical device accessory and/or medicaments, e.g. for self-treatment or home-medication.

### BACKGROUND

Patients suffering from certain diseases like, for example, hemophilia or requiring enzyme replacement therapy have to take regular intravenous (IV) infusions. The infusions often have to be mixed and prepared, sometimes to the specific needs of the patient, (and sometimes a short time before drug administration) which may include reconstitution of the drug powder from multiple vials using an exact amount of sterile liquids like water and/or saline. As this preparation process is typically complex and tedious, it is usually performed by a health care professional in a clinic or pharmacy, potentially using lab equipment.

Generally, administering a medicament by way of infusion may require a rather clean or sterile environment. A patient may therefore have to regularly visit an ambulance or health care center.

Self-medication or home-medication for administering a medicament through infusion or injection is and remains quite challenging but is very attractive for patients thereby avoiding problems and circumstances involved in visiting a health care center. With home- or self-medication a patient or user, e.g. intending to establish a vascular access to a patient's body, may be obliged to use only one hand, which might be rather cumbersome and thus challenging.

In addition, it is often required to establish or maintain a clean and/or sterile environment especially in the field of home-medication or self-medication as well as providing of a clean and sterile storage environment for medicaments and medicament containers, medical device accessory and medical devices.

It is therefore desirable to provide improvements in the field of home medication or self-medication, which allow a user or caregiver to prepare and to administer a medicament by way of injection or infusion. It is further desirable to provide an improved storage and transportation of medical devices, medicaments, medical device accessory and the like components required for home- or self-medication. Furthermore, there should be provided improvements in guiding and assisting a user in conducting or executing numerous steps in the course of preparing medicaments and/or in the course of preparing administering of a medicament, e.g. by way of infusion or injection.

### SUMMARY

In one aspect the present disclosure relates to a portable patient care kit. The portable patient care kit comprises a housing. The housing comprises a first housing component and a second housing component. The second housing component is movably connected to the first housing component. The housing is reconfigurable between a closed configuration and a configuration of use by moving the second housing component relative to the first housing component.

The portable patient care kit comprises at least one compartment provided on an inside of one of the first housing component and the second housing component. The at least one compartment is configured to accommodate an item. The item is selected from at least one of a medical device, a medical device accessory, a medicament container, dressing material, a portable electronic device, an illumination device and an optical device.

The portable patient care kit further comprises a holder. The holder comprises a mount for fastening the item or an external item and to the holder. The holder is connected or the holder is connectable to at least one of the first housing component and the second housing components to protrude from the inside of one of the first housing component and the second housing component when the housing is in the configuration of use.

Hence, the holder is configured to hold at least one item and to keep the respective item in a raised or elevated position relative to at least one of the first and the second housing components. The holder can be arranged, configured or moved into a protruding position, in which the holder protrudes from an inside of at least one of the first housing component and the second housing component when the housing is in the configuration of use. In this way the holder with the mount for the item allows and supports a dedicated holding or fastening of the item in an elevated or raised configuration, which is beneficial for a user or patient when using an item in the preparation of a medicament or in the course of preparing or conducting administering of a medicament.

When in one of the raised position, elevated position or the protruding position, the mount and/or the holder may be separated by a distance from the inside of the first housing component and from the inside of the second housing component. This distance may be larger than an extent of the item configured for fastening to the holder or to the mount. In this way and when the item is fastened to the holder it may be located in a raised, elevated or protruding position or configuration with regard to at least one of the inside of the first housing component and from the inside of the second housing component. In this way and when fastened to the holder and/or to the mount the item may be separated from the inside of the first and the second housing components. In may be in a non-contact position or non-contact relation with the inside of the first housing component and that all the second housing component.

In some examples the holder is also reconfigurable between a protruding of raised configuration and an initial or storage configuration. In the initial or storage configuration the holder may be stored inside the housing. It may be enclosed by at least one of the first and the second housing components or between these housing components, which may form or constitute a closed housing providing an interior volume, which is sealed or closed with respect to the environment. Insofar and when the housing is in the closed configuration the first and the second housing components may mutually complement to form a sealed or closed enclosure for the interior volume of the housing. The first housing component may be then connected or attached to the second housing component in such a way that the first and the second housing components form or constitute the enclosure for the items to be stored or transported inside the housing. The first housing component may sealingly engaged with the second housing component when the housing is in the closed configuration. This way, the interior of the housing can be effectively protected against environmental hazards or influences, such as dust, humidity or other contaminants.

For transferring the housing in the configuration of use the first housing component is to be moved relative to the second housing component; or vice versa. This way, an inside of at least one of the first and the second housing components becomes accessible for a user. Since a compartment for an item is provided on the inside of at least one of the first and the second housing components the user may gain access to the respective compartment and to the respective item as soon as the housing is transferred into its configuration of use. The user may then select and remove an item from a compartment and mount or fix the respective item to the mount of the holder. Concurrently, the holder may be arranged or transferred in the protruding or raised position or configuration so that the item mounted to the mount of the holder is correctly held in a desirable position, e.g. which position is required for or is beneficial for the preparation of the medicament and/or for the preparation of an injection or infusion procedure.

In some examples the housing consists of the first housing component and the second housing component. In further examples the housing comprises an intermediate section located between the first housing component and the second housing component. Here, the first housing component and/or the second housing component may each comprise a rather rigid housing component, e.g. implemented as a housing shell or housing tray. The intermediate section may be connected with both, the first housing component and the second housing component and may constitute or form a link between the first housing component and the second housing component. The intermediate section may comprise or constitute a flexible connector between the first housing component and the second housing component. The intermediate section may allow and support a movability of the first housing component relative to the second housing component. The first housing component may be non-detachably connected to the second housing component by way of the intermediate section. The intermediate section may be implemented as a flexible strap or flexible band interconnecting the first housing component and the second housing component. This way the intermediate section also provides a permanent fastening of the first housing component to the second housing component as well as a movability of the first housing component relative to the second housing component.

According to a further example the holder is detachably connected to at least one of the inside of the first housing component, the inside of the second housing component and an inside of the intermediate section located between the first housing component and the second housing component when the housing is in the configuration of use. There may be provided at least one compartment on an inside of one of the first housing component and the second housing component, which compartment is sized or configured to receive the holder. This way, the holder can be stored inside the housing and can be detachably connected to at least one of the inside of the first housing component, the second housing component and the inside of the intermediate section when the housing is in the configuration of use.

When connected to the housing component or intermediate section the holder may protrude from the inside of the respective housing component or intermediate section. The protruding holder may hinder or prevent to transfer the housing from the configuration of use back into the closed or storage configuration. For this, the holder may have to be detached from at least one of the first and the second housing components and the intermediate section and may have to be stored in a respective compartment of one of the first and second housing components. By storing the holder in a compartment as provided on the inside of at least one of the first and the second housing component it can be securely kept inside and attached to the housing and may no longer block or hinder transferring of the housing from the configuration of use into the closed configuration. Moreover, by providing a storage space for the holder inside the housing the holder can be securely stored inside the portable patient care kit.

According to another example the holder is attachable or is attached to at least one of the inside of the first housing component, the inside of the second housing component and an inside of the intermediate section located between the first housing component and the second housing component. The holder is typically attached to the respective component or intermediate section by at least one joint or hinge. In some examples there may be provided numerous hinges by way of which the holder is pivotably attached to at least one of the first housing component, the second housing component and the intermediate section. The pivotable arrangement of the holder to the inside of the housing may facilitate transferring of the holder from an initial or storage configuration into a protruding or raised configuration. The pivotable arrangement or fastening of the holder to the inside of the housing thus facilitates the use of the portable patient care kit.

According to a further example the holder is fixable in the protruding or radially position by the hinge. When in the protruding position the holder protrudes from the inside of at least one of the first housing component, the second housing component and the intermediate section.

The hinge between the holder and the inside of the housing may be provided with an interlock allowing to fix the holder in a dedicated protruding position relative to the inside of at least one of the first housing component and the second housing component. The interlock may be implemented as a snap lock, which may automatically activate at the end of a pivoting motion during which the holder is moved from the initial or storage configuration relative to at least one of the first and the second housing component into the protruding or raised position. Release of the interlock may automatically take place when a user exerts a respective force to the holder relative to at least one of the first and the second housing component, which user-applied force or torque is above a maximum holding force provided by the interlock.

According to a further example the first housing component and the second housing components are mutually movably interconnected by the intermediate section. The intermediate section may be flexible or pliable. The first and the second housing components may be rigid and/or stiff. The flexibility or pliability of the intermediate section allows and supports a movement of the first housing component relative to the second housing component.

According to other examples the housing may be void of an intermediate section. Then, the first housing component and the second housing component may be directly movably connected relative to each other, e.g. by a hinge.

According to a further example of the patient care kit the first housing component and the second housing component are pivotable relative to each other with respect to a housing pivot axis. The housing pivot axis extends between the first housing component and the second housing component. The housing is transferable from the closed configuration into the configuration of use by pivoting the first housing component relative to the second housing component with respect to the housing pivot axis.

According to further examples of the patient care kit, wherein the housing is provided with an intermediate section between the first and the second housing component a respective pivot axis may extend through the intermediate section or may be formed or constituted by the intermediate section.

A housing with first and second housing components rotatable or pivotable relative to each other with respect to a housing pivot axis provides a well-defined movability of the first and the second housing components. Here, and when transferred into a configuration of use, an outside surface of the first and the second housing component may form or constitute a support, by way of which the opened housing may rest on an external support, e.g. on a table.

According to some examples the housing pivot axis may overlap or may extend through a portion or section of at least one of the first housing component and the second housing component. Hence, a hinge defining the housing pivot axis may be formed or constituted by respective hinge components integrally formed or provided on the first housing component and the second housing component, respectively.

According to a further example of the patient care kit the holder comprises a first leg and a second leg and a longitudinally extending traverse. The first leg is connected to a first longitudinal end of the traverse. The second leg is connected to a second longitudinal end of the traverse. The mount for the at least one item is provided on or in the traverse.

Typically, an end section of the first and the second legs facing away the traverse may be connected to the inside of the housing. In this way, the traverse may be in an upright or protruding configuration relative to the first housing component or relative to the second housing component. Accordingly, and since the mount is located on or in the traverse the mount is elevated and hence the item to be supported or fixed to the mount can be kept in a raised or elevated position or configuration relative to the first housing component and/or relative to the second housing component.

Connecting the traverse of the holder by a first leg and a second leg may improve a stability and rigidity of the holder. It may be thus mechanically stabilized.

According to a further example a first end of the first leg is connected with the first longitudinal end of the traverse. A first end of the second leg is accordingly connected with the second longitudinal end of the traverse. The two legs and the traverse may form a inverted U-profile or inverted U-shaped bracket, when the holder is in the protruding or raised configuration relative to the housing and when the housing is in the configuration of use. This geometric configuration of the holder is of particular benefit since it provides a rather universal handling for a user.

As an example, a user may grip any of the first leg, the second leg and the traverse to transfer the holder from the storage or initial configuration into the protruding or raised configuration. The legs and the traverse may be rigidly attached to each other. In some examples the legs and the traverse are integrally or unitarily formed.

According to a further example the holder comprises a closed frame. The holder may be of rectangular or oval shape. The holder with its frame may define or comprise a planar structure. Hence, the individual holder part or portions, namely the traverse and the two legs may be located in a common plane.

In some examples of the holder the first and the second leg may extend parallel to each other. Also, and when in the protruding or raised configuration the traverse of the holder may extend parallel to a plane defined by a support on which the portable patient care kit is located on and/or the traverse may extend substantially parallel to an inside plane or outside plane of at least one of the first housing component and the second housing component. In some examples and when in the protruding or raised configuration the traverse may extend or protrude upwardly from the housing components, thus facilitating attachment of individual items to the mount of the holder.

It some examples of the portable patient care kit the holder comprises numerous mounts for numerous individual items that may be stored inside the housing or which may be provided externally by a user. Numerous mounts may be located next to each other, e.g. in an overlapping or non-overlapping way on the traverse and/or on the legs. This allows to keep or to hold numerous individual items in a place and well-defined position or configuration for assisting a user in preparing at least one of a medicament and in preparing the administering of the medicament, e.g. by way of injection or infusion.

According to a further example and when the holder comprises a closed frame the holder also comprises a bottom traverse interconnecting a second end of the first leg and a second of the second leg. The bottom traverse may extend substantially parallel to the traverse, which may be considered as a top or upper traverse. The frame may be of a rectangular structure. The first and second legs as well as the traverse and the bottom traverse may extend or may be located in a common plane. Such a planar structure of the frame or holder facilitates its storage inside the housing of the portable patient care kit.

According to a further example a second end of the first leg is pivotably attached to the inside of one of the first housing component, the second housing component and the optional intermediate section via a first hinge. The second end of the second leg is correspondingly pivotably attached to at least one of the inside of the first housing component, the inside of the second housing component and the intermediate section via a second hinge. A twofold hinged connection between the holder and at least one of the first housing component, the second housing component and the intermediate section provides an improved and mechanically stable connection between the housing and the holder. The twofold hinged connection may provide an increased stability of the holder in terms of fixing the holder to the housing as well as in terms of providing a well-defined pivoting and fixing of the holder relative to the housing, e.g. in the protruding or raised configuration.

In some examples and when the holder comprises a closed frame also the closed frame may be connected with at least one of the inside of the first housing component, the inside of the second housing component and the optional intermediate section by way of a first hinge and/or by way of at least two hinges or several hinges.

According to a further example the holder may be pivotably attached to an inside of the housing by way of a film hinge. Here, the holder may comprise a bottom traverse, which may be pivotably connected to the inside of the housing through a longitudinally extending film hinge.

According to a further example of the patient care kit and when the housing is in the configuration of use an outside surface of the first housing component extends substantially parallel to an outside surface of the second housing component. Here, it may be provided that at least one of the first outside surface and the second outside surface is or forms a support surface through which the patient care kit can be positioned on an external support.

In some examples and e.g. when the first housing component is pivotably attached to the second housing component transferring of the housing from the closed configuration into the configuration of use may include a rotation or pivoting motion of the first housing component relative to the second housing component by about 180°. Here and in the closed configuration the outside surfaces of the first housing component and the second housing component may extend substantially parallel to each other comparable with an upper and a lower side of a closed suitcase lying on a support. Pivoting the first housing component relative to the second housing component so as to transfer the housing into the position of use may then include a pivoting of the first housing component relative to the second housing component by about 180° with respect to the housing pivot axis.

Then, the first housing component may reach a position next to the second housing component and both, the first outside surface and the second outside surface may be positioned on a support thereby providing a rather stable, tilt-free position or configuration of the housing when in the configuration of use. In addition, and by transferring the housing into the position of use a contact surface or contact area through which the housing is supported by a support can be increased, thereby stabilizing the position or orientation of the housing on an external support or support structure. This way there can be provided a rather stable and tilt-free arrangement of the housing when in the configuration of use.

According to another example and wherein when the housing is in the configuration of use the holder is in an upright position or is configured to reach an upright position, in which the mount is in an elevated position relative to the inside of each one of the first housing component and the second housing component. In some examples and after having transferred the housing into the configuration of use the holder may either be manually positioned into the protruding or upright position or orientation. In some examples the holder may be mechanically coupled to at least one of the first housing component and the second housing component so as to reach the upright or protruding position relative to at least one of the first and the second housing components when the first housing component has been pivoted or moved relative to the second housing component to reach the configuration of use.

By having the mount in an elevated or raised position relative to the inside of at least one of the first housing component and the second housing component the at least one item, e.g. a medical device, a medical device accessory, a medicament container or a portable electronic device can be kept in a desirable, e.g. in a raised, tilted or elevated position or in a desirable orientation relative to at least one of the first and the second housing components, which position or orientation may be beneficial to prepare a medicament or to prepare or to assist a user in preparing and/or administering of the medicament. Here, and when the mount is in an elevated or raised position the mount may be located at a distance from the inside of at least one of the first housing component and the second housing component. The mount in its entirety may be separated by a distance from the first housing component and the second housing component. This way, also an item fixed or connected to the mount may be separated from the inside of at least one of the first and the second housing components, e.g. by a certain distance. The item may be then located at a respective distance, which may facilitate use or handling of the respective item for preparing or administering the medicament.

According to a further example the patient care kit comprises an armrest connectable or connected with the inside of one of the first housing component and the second housing component. The armrest may comprise a planar-shaped body. The armrest may cover an inside of at least one of the first housing component and the second housing component. A user may place his arm on the armrest for an injection or infusion procedure. The armrest may be initially stored in a sterile or clean environment inside the housing. The planar-shaped armrest may help to increase user comfort and may also help to improve to keep the environment of the patient clean and/or sterile.

By having the armrest connected or connectable to the inside of one of the first housing component and the second housing component a respective housing component connected with the armrest or providing the armrest may be inherently fixed by the user when a user deposits his arm on the armrest. Laying down of an arm on the armrest may inherently immobilize the armrest, which due to its connection with at least one of the first and the second housing components also fixes the respective housing component and hence the housing of the portable patient care kit.

According to a further example the armrest of the patient care kit comprising a body with a base portion connected or connectable with one of the first housing component and the second housing component. The armrest further comprises at least one armrest extension pivotably connected to the base portion and pivotable relative to the base portion between an extended configuration and a storage configuration. Typically, and when in the storage configuration the armrest extension overlaps with the base portion. It may be located on the base portion. When in the extended configuration the armrest extension extends beyond at least one of the first housing component and the second housing component. By way of an armrest extension the overall size of the armrest can be increased to such an extent that it is or becomes larger than the planar size of the respective housing component. In this way, and by having an armrest with a base portion and at least one movable or pivotable armrest extension the armrest can be stored in a rather compact way inside the housing and may be unfolded into the extended configuration so as to provide a comparatively large area for supporting an arm of a user.

In a further example of the armrest the armrest comprises a first armrest extension pivotably connected to a first end of the base portion and further comprises a second armrest extension connected to a second end of the base portion. First and second ends of the base portion may be opposite ends of the base portion. In this way and by having a first and a second armrest extension the dimensions of the armrest in a longitudinal direction, e.g. extending from the first end towards the second end of the base portion of the armrest can be extended by the size of the first armrest extension and by the size of the second armrest extension.

According to a further example the first armrest extension may be further connected with a third armrest extension. The third armrest extension may be connected to an end or end section of the first armrest extension facing away the base portion of the armrest. Likewise, the second armrest extension may be provided or connected with a fourth armrest extension, which may be likewise pivotably connected to an end section of the second armrest extension facing away the base portion. This way there can be provided a multiply folded or multiply foldable armrest, which can be transferred into a storage configuration, in which the armrest only requires a minimum of storage space. By transferring the armrest in its extended configuration, e.g. by unfolding numerous armrest extensions the total size of the armrest can be increased.

Pivot axes between the base portion and armrest extensions as well as between mutually adjoining armrest extensions may extend parallel or perpendicularly to each other. With a parallel extension of numerous pivot axes interconnecting a number of armrest extensions the total size of the armrest in the extended configuration can be extended with regard to a first direction, e.g. extending perpendicular to the numerous pivot axes. With pivot axes of armrest extensions extending at a predefined angle, e.g. at an angle of 90° relative to each other the total size of the extended armrest can be increased in at least two dimensions.

According to a further example the armrest extension is pivotable relative to the base portion with respect to the armrest pivot axis extending parallel or along a side edge or sidewall of at least one of the first housing component and the second housing component. Here, the base portion may entirely cover an inside of one of the first housing component and the second housing component. By pivoting the armrest extension from its storage configuration into the extended configuration the entirety of the armrest extension may extend beyond the respective housing component and may effectively increase the size of the armrest.

The armrest may comprise a padded planar sheet. The armrest may comprise a woven or nonwoven fabric. The armrest may comprise a closed planar structure, e.g. being void of through openings or recesses. By covering the entirety of the inside of e.g. the first housing component it may provide a cover for the compartments exclusively provided in the second housing component when the housing is or reaches the closed configuration. Then, the armrest may form or constitute a closed layer, which gets into an overlapping and closing configuration with the compartments provided or located on the inside of the second housing component. In this way, the armrest may provide twofold function. It may provide an armrest for an arm of a user when the housing is in the configuration of use.

When the housing is in its closed configuration, the armrest, e.g. its padded and closed body may cover the compartment or compartments as provided on the inside of the second housing component. Here, it may effectively seal or close the inside of the second housing component. In this way, the items stored in the compartment or in the compartments may be additionally secured by the armrest covering the compartment or compartments of the second housing components.

According to a further example of the patient care kit the mount is configured to detachably hold a medical device, e.g. an injection device in an upright orientation. The injection device may be an item that is to be stored inside the housing of the portable patient care kit. The mount may be configured to hold the injection device in the upright orientation. This may be beneficial for the preparation of the medicament located inside the injection device or provided in a separate medicament container, such as a vial or bottle.

In some examples the injection device comprises a syringe. The injection device may comprise a syringe equipped with an injection needle. In another example the injection device may comprise a syringe connected or connectable with an infusion line. The upright orientation of the injection device when fixed or held by the mount is beneficial to support an air management inside the injection device.

According to a further example the mount comprises one of a clip and a slot to receive and/or to fix the injection device to the holder. The injection device may comprise a barrel, e.g. of tubular or cylindrical shape. The injection device may further comprise a plunger or stopper movably disposed relative to the barrel. By way of the plunger, e.g. extending into an inside of the proximal end of the barrel, a stopper or respective plunger can be moved or urged in a longitudinal distal direction relative to the barrel in order to expel a liquid medicament located inside the barrel through an outlet of the barrel, which is provided at a distal end of the barrel of the injection device. The clip or slot may comprise a geometry or structure that enables insertion of the barrel of the injection device in longitudinal direction into the clip or slot. With other examples the clip or slot may comprise a pivotable or elastically deformable clip portion, which enables a radially directed insertion of the tubular shaped barrel of the injection device into the clip.

By way of a clip or slot, the injection device can be fixed in an upright orientation to the mount and hence to the holder of the portable patient care kit, particularly when the holder is in the protruding or raised configuration above the first and/or second housing component.

Providing of an open clip or slot may be of further benefit to insert the injection device into the mount while an outlet of the injection device is already connected with an infusion line. The infusion line may easily fit through the opening of the clip or slot.

According to a further example the mount comprises a non-rotating feature configured to non-rotatably engage with a respective portion of the injection device. In some examples the non-rotating feature and hence the mount comprises a protrusion protruding from a sidewall of the mount and being configured to non-rotatably engage with a dedicated portion of the injection device. When the injection device is implemented as a syringe it may comprise a finger flange to support e.g. an index and a middle finger of a user. Here, the flange, typically provided at a proximal end of a longitudinal or tubular-shaped barrel of the injection device or syringe may engage or may circumferentially abut with the protrusion or with numerous protrusions as provided e.g. on the mount. This way, the injection device, e.g. the syringe, can be fixed in a non-rotative manner to the mount. A rather stable mounting or fixing, e.g. non-rotatable fixing of the injection device to the mount and hence to the holder is of particular benefit when the user or patient intends to use some more components or items as provided in one of the compartments of the housing in the course of preparing the medicament and/or in the course of preparing administering of the medicament.

According to a further example the mount is configured to hold and/or to fix at least one of an illumination device and an optical device. The illumination device may be stored in a compartment of at least one of the first and second housing components. The illumination device may comprise a light source. The light source may be configured to illuminate an injection site of a patient, e.g. a portion of an arm located next to the holder and/or located on an armrest of the portable patient care kit. The illumination device may be of particular benefit to illuminate an injection site of a patient and/or to increase a visibility of e.g. the vascular system of the patient.

The light source of the illumination device may comprise one or several light emitting diodes. The light source may be configured to generate light in a rather warm white color. The spectrum of the white color may comprise a color temperature in a range of 1500 K-5000 K. In some examples the color temperature of the light source of the illumination device may be in a range of 2000 K-4000 K. In some examples it may be around 2700 K-3300 K. Such a spectral range or color temperature of the light source is of particular benefit to enhance visibility of the vascular system of the patient.

In some examples the illumination device comprises a gooseneck and a plug. The light source may be mechanically connected to the holder by the plug engaging a socket of the mount as provided on the holder. The light source may be connected to the plug via the gooseneck. According to further examples the illumination device comprises a switch, which is manually actuatable or controllable by a user. This way, a user may selectively control or manipulate operation of the light source of the illumination device. The illumination device may be reconfigurable, e.g. by the switch. Hence, a light intensity or light color of the light source may be modifiable by a user when actuating or manipulating the switch.

By way of a gooseneck the position or orientation of the light source relative to the plug can be modified by a user. This way, a direction of a light or light beam generated by the light source can be easily modified by a user.

With a further example the mount comprises a socket to receive a complementary shaped plug of the illumination device or optical device. The illumination device may be detachably connected to the socket of the mount of the holder by the plug. Here, the plug and the socket provide a mechanical connection between the illumination device and the mount and hence between the illumination device and the holder of the patient care kit.

In some examples the illumination device comprises an own energy source, such as a battery in order to provide electrical power for the light source. Hence, the illumination device may operate stand alone. In other examples the socket of the mount and the plug of the illumination device are configured as an electrical power connector through which electrical power or electrical energy can be transmitted from the socket of the mount to the illumination device. Here, the illumination device may be void of an own power supply or energy source. It may be provided with electrical power through the socket of the mount of the holder. Insofar, the holder may provide a twofold function. First of all, it may provide a mechanical fixture and fastening for the illumination device e.g. in a raised or elevated position or orientation. Second, the mount may provide electrical energy for operating the illumination device.

In a further example the optical device may comprise a magnifying glass or a magnifier. The optical device may be implemented in a similar fashion as the illumination device. The optical device may also comprise a plug and a gooseneck or gooseneck portion by way of which the magnifier is connected to the plug. The plug of the optical device may be inserted into a socket as provided on the holder of the portable patient care kit. Accordingly, the magnifying glass of the optical device can be oriented in a rather arbitrary manner in accordance to the preferences or demands of the user. By way of the optical device, e.g. implemented as a magnifying glass, an injection or infusion site of the patient can be magnified, thus facilitating the process of piercing or penetrating the patient's skin, e.g. by an injection or infusion needle.

According to a further example the mount comprises a needle unit holder configured to detachably hold and/or to detachably fix a needle unit. The needle unit holder may be also denoted as a needle mount. The needle unit may comprise an injection needle or an infusion needle. In some examples the needle unit may comprise a so-called butterfly needle. The needle unit may be provided with an injection needle or infusion needle and a handle rigidly or flexibly connected to the needle and allowing to hold and/or to insert the needle into biological tissue. The needle unit may further comprise a needle cap covering the tipped end of the needle. The needle cap may be detachably connected with the needle unit. It may be detached from the needle so as to expose the tipped end of needle prior to use the needle for piercing the skin of the patient.

The needle mount allows to at least temporarily fix the needle unit in a well-defined position, typically at the traverse of the holder in a raised or elevated position above the first and/or second housing components of the patient care kit and hence above an armrest of the patient care kit. By fixing the needle unit to the respective mount the needle unit can be provided in a well-defined position, which is beneficial for a user to grip the needle in the course of preparing an injection or infusion procedure.

The mount for the needle unit holder may comprise a sleeve to receive at least a portion of the needle unit. The sleeve or the mount may be configured to fix the needle unit via the needle cap of the needle unit. Hence, the needle unit may be inserted with its needle cap into the needle mount and in particular into the sleeve of the needle mount. The needle cap may be inserted in longitudinal direction into the needle mount. The needle cap may be fixed in the needle mount by a respective fastener, which enables insertion of the needle cap into the mount along a first direction and which is operable to prevent removal of the needle cap in an opposite direction. This way, the needle mount may provide a fixing of the needle cap while a user may exert a removing force onto the needle unit, e.g. via the handle of the needle unit, to separate the needle unit from the needle cap.

Here, the needle cap may remain fixed to the needle mount and the needle may be separated from the needle cap, simply by tearing the needle unit in a second direction opposite to the first direction, along which the needle unit is attached of fixed to the needle mount. The fixing of the needle cap to the needle mount allows for a single-handed separation of the needle cap from the needle unit. A user may take one hand and insert the needle unit with the needle cap into the needle mount along the first direction until receiving a fixing position. Then and when reaching the fixing position the needle cap is inherently secured against withdrawal or removal from the needle mount in a second direction opposite to the first direction. The user may then apply a separation force onto the needle unit, e.g. via the handle of the needle unit, thereby exerting a relative force between the needle cap and the needle unit. Since the needle cap is prevented or blocked from moving along the second direction the force as exerted by the user onto the needle unit in the second direction leads to a separation of the needle unit from the needle cap. Accordingly, the user may simply retract the needle unit from the needle mount, thereby inherently separating the needle unit from the needle cap, which separation is necessary to expose the tipped end of the needle for conducting injection or infusion.

When the needle cap reaches a fixing position inside the sleeve of the needle mount the needle cap is secured against a respective removal in a second, hence opposite direction. After a retraction and hence separation of the needle unit from the needle cap the needle cap, e.g. made of a plastic material, may be easily deformed radially inwardly because it's interior is no longer occupied by the needle unit. Hence, a distal end of the needle cap, which may protrude from the needle unit holder can be gripped by a user.

The user may then apply a respective force onto the needle cap along the first direction. Since the needle unit no longer occupies an interior of the needle cap the needle cap may slightly deform in response to such a user-applied force and may thus slide out of the sleeve of the needle unit holder along the first direction. The needle unit holder may then be ready for holding or fixing of another needle unit.

According to a further example the mount of the holder is configured to removably receive a portable electronic device. The mount may be particularly configured or designed to receive and/or to hold or to fix a personal portable electronic device, such as a smart phone, a tablet computer or a smart watch. The mount may be thus implemented as a device mount. It may be particularly configured to keep and/or to fix the portable electronic device in a well-defined orientation relative to the holder of the portable patient care kit. The device mount may comprise a first and a second guiding structure, e.g. provided on an inside of the first leg and the second leg of the holder, respectively. The guiding structures may each comprise a guiding profile operable or sized to receive a housing of the portable electronic device. The guiding structures or guiding profiles may be oriented at a predefined angle relative to the elongation of the respective leg. This may allow and support a well-defined inclined or tilted orientation of the portable electronic device relative to the plane or orientation of the holder.

The device mount is of particular benefit to keep the external electronic device in an upright, inclined or tilted orientation, which orientation is beneficial for a good visibility of e.g. a display of the portable electronic device. A user installing the portable electronic device into the device mount of the holder in the protruding or raised configuration will be enabled to easily read information provided on the display of the portable electronic device. The portable electronic device may be provided with user instructions or guiding instructions and may be provided with an app or software program that is operable to assist a user in using or preparing at least one of the portable patient care kit and the at least one item stored therein.

According to a further example of the patient care kit at least one of the first housing component and the second housing component comprises a receptacle to receive an inlay. The inlay comprises the at least one compartment. The inlay may cover or may occupy an entirety of the inside of the respective housing component. The inlay may comprise at least one or several recesses forming the at least one or several compartments, respectively. The inlay may be implemented as a tray. The inlay may be implemented as a blow-molded component. It may comprise a rather thin plastic material, which comprises numerous compartments, which are adapted in shape and structure to the items to be stored and/or to be fixed therein.

The at least one compartment may be complementary shaped to an item to be stored and/or fixed therein. The at least one compartment may be formed to frictionally and/or to positively engage with the item to be stored or fixed therein. The item when arranged in a compartment may be clip-fixed or snap fitted with the compartment, e.g. with the respective recess formed in the inlay or tray.

According to some examples the inlay or tray is at least in portions transparent or partially transparent.

The inlay may be configured in accordance to an intended equipment or equipment configuration of the portable patient care kit. Some patient care kits may be configured to provide storage and assistance for injecting a medicament provided in a medicament container of a particular size A. Other portable patient care kits may be configured to provide storage space for medicament containers of a different size, e.g. of a size B.

Here and by having the compartments for the respective items detachably connected with e.g. a second housing component, a reconfiguration of a portable patient care kit can be easily provided by simply changing the first inlay configured for fixing or receiving of a first item by a second inlay configured or shaped to receive and/or to fix a second item of different size.

According to a further example the patient care kit comprises an illumination module arranged inside at least one of the first housing component and the second housing component. The illumination module comprises a number of individual light sources distributed across a surface of the illumination module and overlapping with a position of the at least one compartment. The illumination module may of particular benefit to illuminate individual compartments of the portable patient care kit when the housing is in the configuration of use. In some examples the illumination module comprises a first and a second light source, wherein the first light source spatially overlaps with a first compartment and wherein the second light source overlaps with a second compartment.

The illumination module may be located between a bottom of the respective housing component and the inlay. Insofar, the light sources of the illumination module may be implemented to illuminate the respective compartments from below. With a material of the inlay of transparent or semitransparent type respective illumination effects can be provided by the individual light sources of the illumination module.

The light sources and/or the illumination module may comprise an illumination controller, which is connected to the individual light sources of the illumination module. By way of an illumination controller the individual light sources can be selectively switched on or off. The illumination controller may be further operable to control a color and/or or an intensity of the individual light sources as well as a temporal variation of activating or deactivating individual light sources.

According to a further example the illumination module comprises a communication interface to communicate with an external electronic device operable to selectively control operation of the individual light sources of the illumination module. In this example the illumination module may be void of an own illumination controller. Here, a control of the individual light sources may be provided or conducted by the external electronic device. The communication interface may comprise a wired connection between the illumination module and the external electronic device. Here, the communication interface may comprise a transmission line provided with a plug complementary shaped to respective socket of the external electronic device.

In other examples the communication interface may be implemented wirelessly. Here, the illumination module with its communication interface may be configured to wirelessly communicate with the external electronic device. The communication interface may comprises a RF transceiver, e.g. implemented in accordance to wireless communication standard, such as RFID, Bluetooth, Blootooth Low Energy (BLE) or NFC.

According to a further example the patient care kit comprises a battery connectable with at least one external electronic device or connected to one of the mount as described above. The battery may be further connected to a socket or plug allowing to recharge the battery when connecting the socket or plug to an external power supply. Insofar, the battery may be implemented as a rechargeable battery. The battery may be provided in one of the first housing component and the second housing components. It may be located outside the holder. The battery may be connected by an electrical conductor with at least one of the mounts.

In some examples the battery is connected to a mount, which is configured to support the illumination device as described above. Here, the socket of the mount may comprise a power connector electrically connected to the battery. This way, and when attaching the illumination device to the respective mount there may be established an electrical connection between the illumination device and the battery of the patient care kit.

In another example and when the battery of the patient care kit is connected with an external electronic device the battery of the patient care kit can be charged by the external electronic device. Vice versa, the battery of the patient care kit may provide a power bank for charging a battery of the external electronic device. The battery of the patient care kit may be electrically connected to an external power supply either by way of a wired connection, e.g. via a transmission line and via a respective plug and socket connection. In other examples the battery of the patient care kit may be electrically coupled in a power transmitting manner wirelessly with an external charging device. Here, even the external electronic device may provide a wireless charging of the battery of the patient care kit, e.g. via an inductive wireless coupling and/or via a NFC wireless coupling.

In another example the patient care kit may be void of an own battery. Then and when equipped with an illumination module the electric power for operating the illumination module may be provided by the external electronic device. Here, the external electronic device may be coupled with the illumination module in an electrical power transferring manner, e.g. by way of a transmission line and a respective plug and socket connection or wirelessly, e.g. by an inductive- or NFC-type coupling.

The electronic coupling between the external electronic device and the illumination module is of particular benefit when the electronic device provides instructions to a user or assists a user in operating at least one of the portable patient care kit and one or numerous of the items stored therein. Here, the external electronic device and/or the illumination module itself may be operable to visibly mark a compartment equipped with a particular item, which according to instructions as provided by the external electronic device should be taken or operated by a user of the portable patient care kit. In this way, a user can be rather intuitively guided through a sequence of steps necessary to prepare a medicament for injection or infusion and/or intended to prepare or conduct numerous preparation steps for an infusion or injection.

In particular, a user may be instructed to take a first medicament container out of a particular compartment. Respective information will be illustrated on the display of the external electronic device. Concurrently, the compartment equipped with the respective medicament container will be illuminated or visibly marked by the illumination module. In a subsequent step the external electronic device may instruct a user to take a medical device accessory, such as a needle unit out of a respective compartment of the portable patient care kit. Then, the respective compartment equipped with such a needle unit may be illuminated accordingly. In the same way, the user may be instructed to take a medical device, such as an injection device, e.g. a syringe out of a respective compartment and so on.

In another example the portable patient care kit comprises numerous mounts some which are connected, fixed or integrated into the holder of the portable patient care kit. The patient care kit may comprise at least or several of the following mounts: the medical device mount, the mount for the illumination device or for the optical device, the mount for a needle unit and/or the mount for an external electronic device.

In further examples the portable patient care kit is equipped with the items to be stored in the at least one compartment as provided on an inside of each one of the first and the second housing components. Hence, the portable patient care kit comprises at least one or several of the following: a medical device, an injection device or infusion device, e.g. in form of an injection or infusion device, a medical device accessory, such as an infusion line, an injection needle or an injection needle unit, a medicament container, such as a barrel filled with a liquid or lyophilized medicament, a container for a liquid, such as water for injection, dressing material, such as plasters or bandages, the illumination device, such as a torch or an LED-operated gooseneck lamp and/or an optical device, such as a magnifying glass or a magnifier.

The medicament container may be implemented as a vial, as an ampoule or carpule or cartridge. It may comprise a pierceable septum at an outlet configured to pe penetrated by a tipped cannula or needle. A liquid medium located inside the medicament container may be thus withdrawn vie the cannula or needle either by suction or by increasing an inside pressure and thus by expelling an amount or dose of the liquid medium.

The optical device may comprise a magnifying glass, e.g. provided with a plug to be connected to a suitable mount or socket as provided on the holder. The magnifying glass may be movably connected to the plug or mount via a gooseneck portion, which allows to arrange and/or to oriented the magnifying glass in a desired position and/or orientation for magnifying a portion of the skin of the patient that should be pierced

The portable electronic device may not be part of the portable patient care kit. It may be implemented as a personal portable electronic device, such as a smartphone or tablet computer. In another example the portable electronic device may be part of the portable patient care kit. It may be also stored in a compartment as provided on an inside of one of the first housing component and the second housing components.

In some examples the portable patient care kit is readily provided with numerous items. It may be prepacked with a medicament container containing a medicament therein. It may be prepacked with a medical device, such as an injection device, e.g. a syringe. It may be readily provided or prepacked with medical device accessory, such as an infusion line, an injection needle or infusion needle. It may be also prepacked with bandages or dressing material. It may also comprise an may be prepacked with the illumination device and the optical device as described above.

According to a further aspect the present disclosure relates to a method of preparing a medicament and/or of preparing administering a medicament to a patient. The method comprises the step of using a portable patient care kit as described above. Insofar, all features, effects and benefits as described above in connection with the portable patient care kit equally apply to the method of preparing a medicament or preparing medicament administering.

In some examples the method comprises the step of transferring the housing of the portable patient care kit from a closed configuration into the configuration of use by moving the first housing component relative to the second housing component. Optionally, a closure of the housing may have to be opened before the respective housing components are moved relative to each other. Here, e.g. a zipper interconnecting a sidewall or an edge of the first housing component with a sidewall or edge of the second housing component may have to be operated by a user before moving the first and the second housing components relative to each other.

In a further step a user may optionally transfer the holder of the portable patient care kit into the protruding or raised configuration.

In a further step at least one item as stored in the at least one compartment of the housing is removed from the compartment and is positioned and/or fixed to a mount as provided on the holder.

When the item, e.g. an injection device a device accessory or a medicament container is suitably mounted on the mount and hence on the holder in the protruding or raised configuration the respective item can be kept in an elevated position relative to the housing of housing components thus facilitating preparation of a medicament, e.g. mixing of a medicament and thus preparing the medicament for infusion or injection.

Optionally and in a further step an armrest may be used and/or unfolded and a user may place his arm on the armrest in preparation of an infusion or injection procedure. In a further optional step the method may comprise installing an illumination device on the mount and activating the illumination device in order to illuminate a portion of the arm or biological tissue of the patient that should become subject to a piercing.

In a further optional step the method comprises taking of a needle unit out of a compartment or pocket of the portable patient care kit and fixing the needle to a needle unit holder as provided on the holder of the patient care kit.

In a further step of preparing at least one of an injection or infusion procedure the needle unit may be retracted or removed from the needle unit holder thereby automatically separating the needle unit from the needle cap.

In another example the method may comprise the step of attaching or mounting an external electronic device to a device mount of the holder. The method may further comprise the step of initiating or starting a software routine or software app on the external electronic device for assisting or guiding a user in executing at least one of preparing of the medicament or in preparing of at least one of an infusion or injection procedure. In subsequent steps the external electronic device may provide readable, visible or audible instructions to the user to conduct at least one or numerous of the above steps.

In another example the external electronic device is electronically coupled with an illumination module of the portable patient care kit. The illumination module comprises a first light source and a second light source, wherein the first light source overlaps with a first compartment and wherein the second light source overlaps with the second compartment.

The first and the second compartments are then separately and/or consecutively illuminated and hence visibly marked by the first and the second light sources of the of the illumination module, respectively.

Here and in accordance to the individual user assistance steps as illustrated or indicated by the external electronic device individual compartments of the portable patient care kit will be concurrently visibly marked by the illumination module. This type of marking can be controlled by the external electronic device in accordance to and/or corresponding with the readable, visible or audible instructions provided to the user through the external electronic device.

Generally, the scope of the present disclosure is defined by the content of the claims. The transportation device is not limited to specific embodiments or examples but comprises any combination of elements of different embodiments or examples. Insofar, the present disclosure covers any combination of claims and any technically feasible combination of the features disclosed in connection with different examples or embodiments.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container”. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(w-carboxyheptadecanoyl)-des(B30) human insulin and B29-N-(w-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom. Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

### BRIEF DESCRIPTION OF THE DRAWINGS

In the following, examples of a patient care kit and its use will be described in greater detail by making reference to the drawings, in which:

FIG. 1 shows an example of a portable patient care kit,

FIG. 2 shows holder of the patient care kit and numerous items to be fixed thereto,

FIG. 3 is an enlarged view of a part of the holder,

FIG. 4 shows the mounting or fixing of an injection device to a mount of the holder,

FIG. 5 shows the injection device mounted to the respective mount of the holder in an enlarged view,

FIG. 6 shows a needle unit fixed to another mount of the holder,

FIG. 7 shows the needle unit detached from the mount with a needle cap remaining fixed to the mount,

FIG. 8 shows the removal of the needle cap from the mount,

FIG. 9 shows another configuration of a needle unit before fixing to a needle unit holder,

FIG. 10 shows the needle unit fixed to the needle unit holder,

FIG. 11 shows the needle unit detached from the needle unit holder and detached from the needle cap, and

FIG. 12 shows the removal of the needle cap from the needle unit holder,

FIG. 13 shows a further configuration of the needle unit holder,

FIG. 14 shows a cross-section through the needle unit holder when engaged with a needle,

FIG. 15 shows a further example of a needle cap fixed in the needle unit holder,

FIG. 16 shows a perspective illustration of the portable patient care kit in the closed configuration,

FIG. 17 shows a further example of the portable patient care kit with an injection device equipped with an infusion line before fixing or mounting to the holder of the portable patient care kit,

FIG. 18 shows the example of FIG. 17 when the injection device and the infusion line are fixed to the holder of the patient care kit,

FIG. 19 is a block diagram of numerous electric or electronic components of the portable patient care kit,

FIG. 20 schematically shows the arrangement of an illumination module and an inlay in the second housing component of the portable patient care kit in connection with a portable electronic device,

FIG. 21 shows a further example of a holder of the portable patient care kit equipped with an electronic device holder before mounting the external electronic device,

FIG. 22 shows an external electronic device connected to the illumination device and fixed by the device holder of the holder in a well-defined orientation,

FIG. 23 shows another example of the portable patient care kit, and

FIG. 24 is a flowchart of a method of preparing a medicament and/or of preparing an injection or infusion procedure thereby making use of the portable patient care kit.

### DETAILED DESCRIPTION

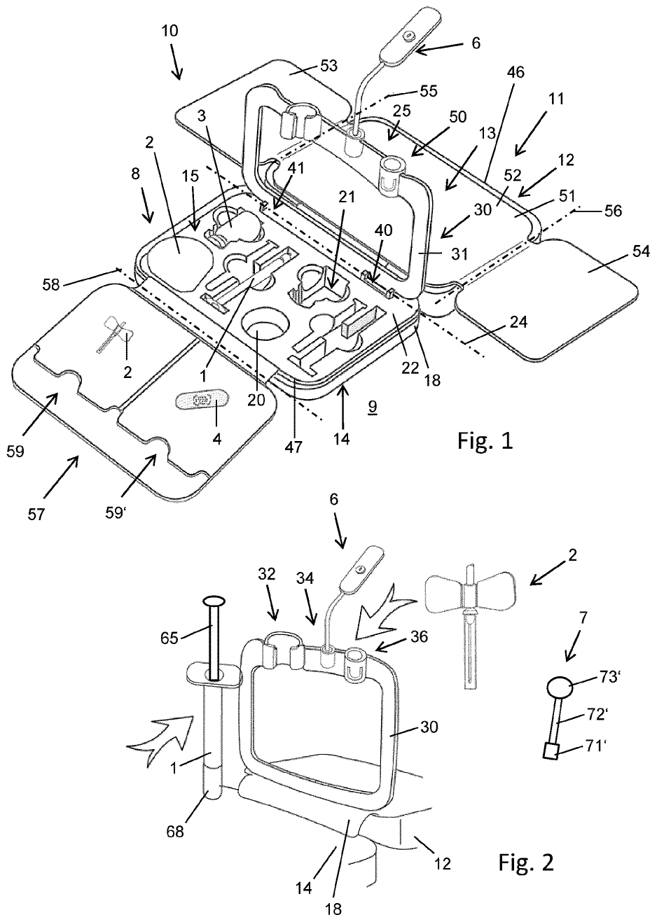

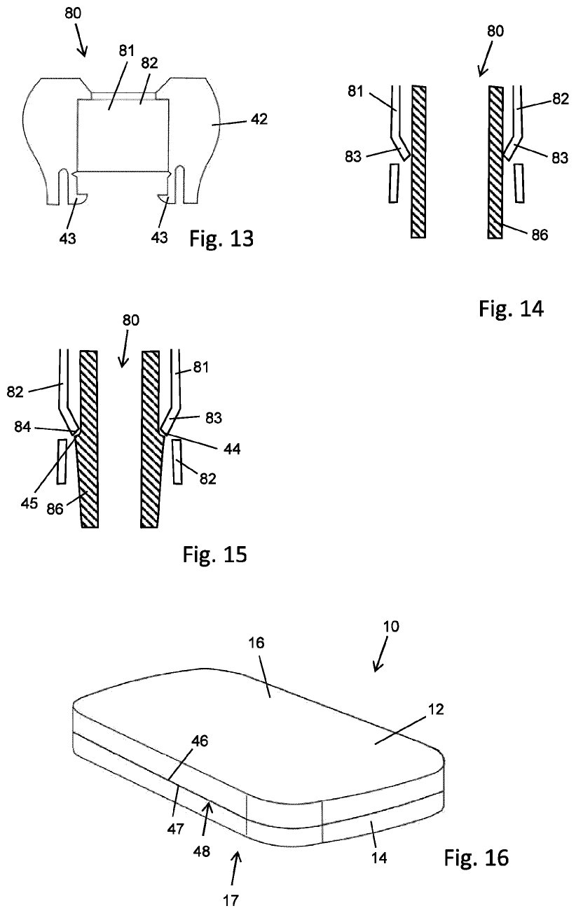

In FIGS. 1-22 numerous examples of a patient care kit are illustrated. The patient care kit 10 comprises a housing 11, which is either in a closed configuration as shown in FIG. 16 or which is in a configuration of use as e.g. shown in FIG. 1. The housing 11 comprises a first housing component 12 and a second housing component 14. Optionally, the housing 11 comprises an intermediate section 18 located between the first housing component 12 and the second housing component 14. The first housing component 12 and the second housing component 14 are movable relative to each other when the housing is in a release configuration. In a closed configuration the first housing component 12 and the second housing component 14 may be locked together. Here, a fastener 46 of the first housing component 12 may be mechanically engaged with a complementary shaped counter fastener 47 as provided on the second housing component 14. The fastener 46 and the counter fastener 47 may be part of a zipper 48, which enables a rather stable and long-lasting releasable connection between the first and the second housing components 12, 14 as well as a rather easy and intuitive handling of a respective closure.

Apart from a zipper solution it is also conceivable that the two housing components 12, 14 are interlocked by any other type of mutually corresponding locking and counter locking features as provided on the first housing component 12 and the second housing component 14, respectively.

The first housing component 12 is pivotally attached to the second housing component 14 with respect to a housing pivot axis 24. Along the pivot axis 24 the first and the second housing component 12, 14 may be undetachably connected. The intermediate section 18, which may be made of a pliable or bendable material and which is permanently and undetachably connected to the first housing component 12 and to the second housing component 14 may overlap with or may form the housing pivot axis 24. The intermediate section 18 may thus form or constitute the housing pivot axis 24.

For transferring the housing 11 from a closed configuration as shown in FIG. 16 into an open configuration or configuration of use, as e.g. illustrated in FIG. 1 the fastener 46 and the counter fastener 47 have to be disengaged so that the housing components 12, 14 remain only connected in the region of the intermediate section 18 and/or by the housing pivot axis 24. The pivot axis 24 and/or the intermediate section 18 may form a hinge by way of which the housing 11 can be transferred from the closed configuration into the configuration of use by pivoting, e.g. the first housing component 12 relative to the second housing component 14 with respect to the housing pivot axis 24 by about 180°.

The first housing component 12 comprises an outside surface 16. The housing component 14 comprises an outside surface 17. In the closed configuration the surface normal of the outside surfaces 16, 17 face in opposite directions. In the configuration of use the outside surfaces 16, 17 are arranged next to each other and their respective surface normals may extend substantially parallel to each other. As further illustrated in FIG. 1 and when in the configuration of use the outside surfaces 16, 17 are configured to support the housing 11 on a support 9, e.g. provided by a tabletop or by a floor.

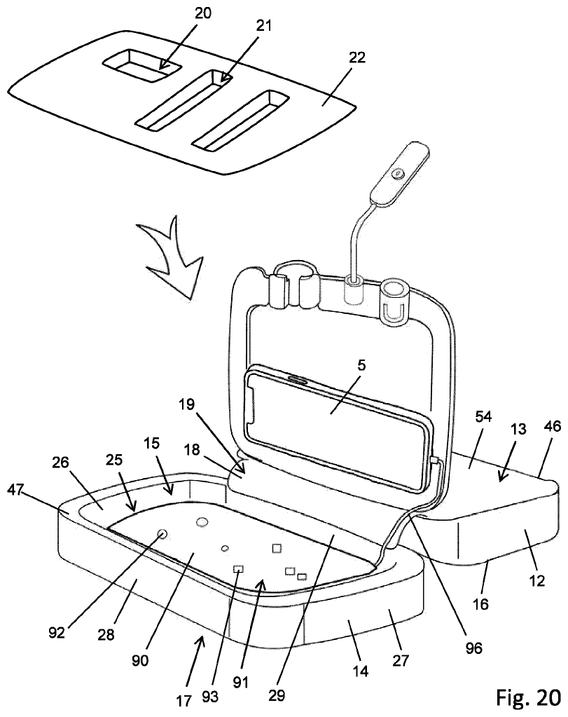

The housing component 12, 14 may each comprise a kind of a tray configuration or a kind of a shell. The housing components 12, 14 may comprise a closed and rigid housing structure. The housing components 12, 14 may be made from a rather rigid plastic material or a metal. The housing components 12, 14 may be shaped rather identical. In some examples they may be shaped differently. However, and as shown in FIG. 20 with the example of the second housing component 14 the housing components 12, 14 each comprise a receptacle 25 being open towards the top and being closed by a bottom (not illustrated) but e.g. provided by or coinciding with an inside of the outside surfaces 16, 17. Hence, the receptacle 25 may be confined by a planar-shaped bottom, such as the outside surface 16, 17.

The receptacle 25 may be further enclosed by a sidewall structure. The sidewall structure of the presently illustrated example comprises a circumferential structure of constant height. The sidewall structure comprises a first sidewall 26 or sidewall section and an oppositely located second sidewall 27 at an opposite longitudinal end of the second housing component 14. The first and second side walls 26, 27 are interconnected by a lateral side walls 28, 29. The sidewalls 26, 27 are located between the longitudinal ends of the sidewalls 28, 29; and vice versa. The sidewall structure constituted by the sidewalls 26, 27, 28, 29 may be integrally formed. It may be single pieced. The housing components 12, 14 may be unitarily formed or may comprise a single piece also including the closed bottom.

The second housing component 14 is configured to provide numerous compartments 20, 21 to receive and/or to fix an item 8, such as a medical device 1, medical device accessory 2, a medicament container 3, dressing material 4, a portable electronic device 5, an illumination device 6, an optical device 7 or some other component, parts or accessory required by a user for conducting or preparing a medicament for injection or infusion and/or for preparing at least one of an injection or infusion procedure.

As particularly illustrated in FIG. 20 the compartments 20, 21 may comprise a recess in an inlay 22 or tray structure, which is configured to be positioned inside the receptacle 25 of the second housing component 14. The inlay 22 may occupy the interior volume as provided or enclosed by the second housing component 14. Optionally, there may be provided an illumination module 90 beneath the inlay 20. The illumination module 90 may be equipped with at least two light sources 91, 92, which may be arranged in a spatially distributed manner on an upper surface 91 of the illumination module 90, thereby facing a lower side of the inlay 22, which is arranged on top or across the illumination module 90.

When in the configuration of use as shown in FIG. 1 the compartments 20, 21 are accessible on the inside 15 of the second housing component 14. Also, an inside 13 of the first housing component 12 is accessible and visible by a user. The first housing component 12 is currently equipped with an armrest 50. The armrest 50 comprises a body 51, which may comprise a padded structure. The body 51 may comprise a base portion 52, which fits in size with the size of the receptacle 25 of the first housing component 12. Hence, the armrest 50 and in particular the base portion 52 of the armrest 50 may entirely occupy the inside volume of the first housing component 12.

The armrest 50 further comprises a first armrest extension 53 and a second armrest extension 54. The first armrest extension 53 is pivotally connected to the base portion 52 with respect to a first pivot axis 55. The second armrest extension 54 is pivotally connected to the base portion 52 with respect to a second pivot axis 56. In the presently illustrated example the two pivot axes 55, 56 are separated along a longitudinal direction of the first housing component 12. The pivot axes 55, 56 overlap or substantially align with a top and a bottom sidewall 26, 27 of the first housing component 12. The armrest extensions 53, 54 can be pivoted relative to the base portion 52 between an extended configuration as shown in FIG. 1 and a storage configuration as indicated in FIG. 20. In the storage configuration the armrest extensions 53, 54 substantially overlap with the base portion 52. In the extended configuration the armrest extension 53, 54 extend beyond the sidewall or sidewalls sections of the first housing component 12. They may protrude outwardly from the sidewall structure of the first housing component 12.

The second housing component 14 is connected with the sidewall or sidewall portion 29 of the first housing component 12. The opposite lateral sidewall portion 28 is provided with a foldable flap 57. The foldable flap 57 is pivotable relative to the second housing component 14 with respect to a longitudinal flap axis 58, which extends substantially parallel or overlaps with the sidewall portion 28. The flap 57 may comprise one or several pockets 59, 59′, which may be used to store various items 8, such as dressing material 4 or medical device accessory 2, such as injection needles or needle units.

The portable patient care kit 10 is further provided with a holder 30. The holder 30 may comprise a closed frame 31 or be implemented as a frame 31. The holder 30 may be pivotable or may be detachably connected with at least an inside 13, 15 of the first or second housing components 12, 14. The holder may be also attached or connected to an inside 19 of the intermediate section 18.

The holder 30 comprises numerous mounts 32, 34, 36, 38 to keep or to hold an item 8. The item 8 may be an item that is stored in one of the compartments 20, 21. The item 8 may be also an external item, such as a portable electronic device 5, which is not intended to be stored inside the housing 11 but which may be provided by a patient or user separately.

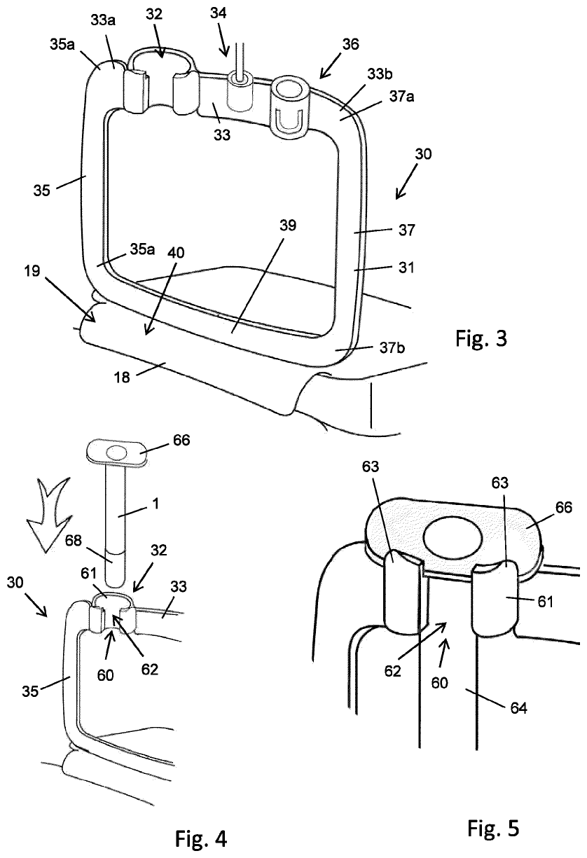

As shown in FIG. 3, the holder 30 comprises a traverse 33, a first leg 35 and a second leg 37. Optionally, the holder 30 further comprises a bottom traverse 39 extending substantially parallel to the traverse 33. The two legs 35, 37 extend substantially parallel to each other. The holder 30 may comprise a rectangularly shaped frame 31, which is undetachably fastened to at least one of the first and the second housing components 12, 14. In the configuration of FIG. 1 the holder 30 is pivotally connected to the inside 15 of the second housing component 14. It may be undetachably connected to the inlay 22.

The holder 30 may be pivoted relative to the housing 11 by way of a hinge 40. There may be provided a first hinge 40 and a second hinge 41 by way of which e.g. the bottom traverse 39 is pivotally attached to the first or second housing components 12, 14. By way of the hinged attachment of the holder 30 to the housing 11 the holder can be moved from an initial or storage configuration into an upright, protruding or raised configuration, the latter of which being shown in FIG. 3. In the raised or upright configuration the traverse 33 is at an elevated position relative to the inside 13, 15 of the first and the second housing components 12, 14. The hinge 40, 41 may be arranged on or in the intermediate section 18. It may also coincide with a hinge or pivot axis by way of which the first and second housing components 12, 14 are mutually pivotally connected.

When in an initial or storage configuration the holder 30 may be pivoted towards or onto the inside 15 of the second housing component 14. The flap 57 may be then located between the holder 30 and the compartments 20, 21 or the inlay 22 providing these compartments 20, 21. In this way and as long as the holder 30 is in the storage configuration the items 8 stored or arranged inside the compartments 20, 21 are secured against an uncontrolled release. Similarly, and when the first housing component 12 is provided with the planar-shaped armrest 50 also the armrest may prevent an uncontrolled release of numerous items 80 from the recesses or compartments 20, 21 as long as the housing 11 is in the storage configuration.

The bottom traverse 39 of the holder 30 may be provided only optionally. The side legs 35, 37 are connected with opposite longitudinal ends 33a, 33b of the traverse 33. In detail, a first end 35a of the first leg 35 is connected with a first longitudinal end 33a of the traverse 33. A first end 37a of the second leg 37 is connected with a second longitudinal end 33b of the traverse 33. Oppositely located second ends 35b and 37b of the first and the second legs 35, 37 may be interconnected with opposite longitudinal ends of the bottom traverse 39.

In another example, wherein the holder 30 is void of a bottom traverse 39, the second ends 35b, 37b of the legs 35, 37 may be directly pivotally attached to the housing 11, e.g. by a first hinge 40 connected to the second end 37b of the second leg 37 and by another hinge 41 connected to the second end 35b of the first leg 35.

The holder 30 is equipped with numerous mounts 32, 34, 36, 38. The mount 32 is implemented as a mount for a medical device 1. The medical device 1 may comprise or may be implemented as an injection device, such as an injector. As indicated in FIGS. 2, 4 and 5 the medical device 1, may be implemented as a syringe. The syringe comprises a tubular-shaped barrel 64 to receive a volume of a liquid medicament. The injection device 1 may be further equipped with a plunger 65 movably disposed inside the barrel 64. The proximal end of the barrel 64 may be provided with a radially outwardly extending finger flange 66. A distal end of the barrel 64 may be provided with an outlet 68. The outlet 68 may be connected to or may be connectable with at least one of an injection needle and an infusion line 88.

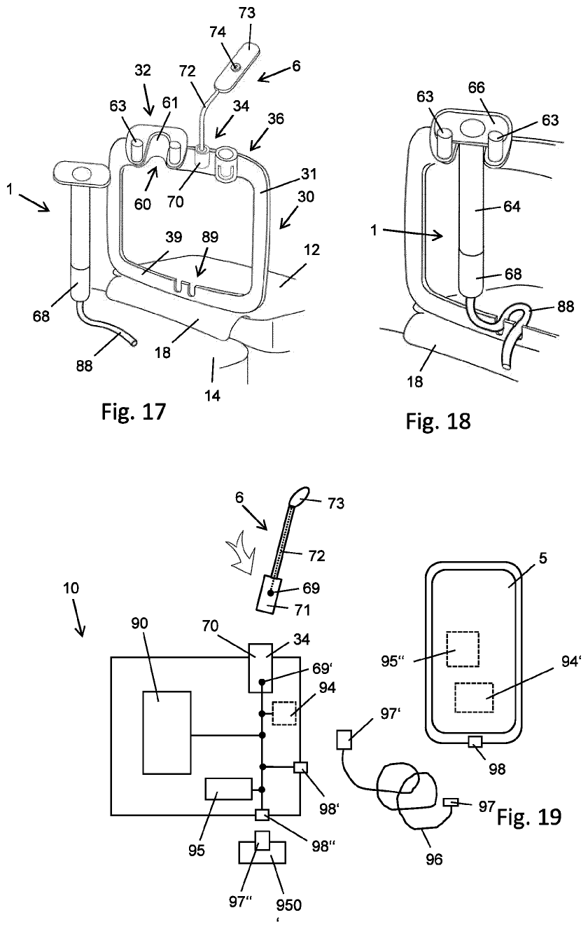

The mount 32 comprises a slot 60 sized to receive the tubular-shaped barrel 64 therein. The slot 60 comprises a tubular-shaped sidewall 61, which is integrally formed with the traverse 33 or which is provided as a separate part connected or connectable to the traverse 33. The sidewall 61 is further provided with a longitudinal and radial through opening 62, thus forming a slit extending through the tubular-shaped sidewall 61. The medical device 1 may be configured for insertion into the slot 60 along a longitudinal direction. Eventually, the outlet 68 of the medical device 1 may be connected with an infusion line 88 as e.g. shown in FIGS. 17 and 18. The infusion line 88 may easily fit through the opening 62. Alternatively, the opening 62 may be sized to receive the barrel 64 therethrough in radial direction.

Hence, the opening 62 may be formed as a clip feature allowing to receive the tubular-shaped barrel 64 along a radial mounting direction. The sidewall 61 may be provided with a single or with numerous protrusions 63 pointing upwardly and thus representing a symmetry breaking feature at an upper or proximal end of the sidewall 61. The protrusions 63 are shaped to form a circumferential abutment or circumferential stop face for the finger flange 66 or a respective radial symmetry breaking feature of the medical device 1.

In this way the protrusions 63 form or constitute an anti-rotation feature, which serves to prevent a rotation of the barrel 64 when the medical device 1 is inserted or received in the mount 32.

As shown in FIGS. 17 and 18, the holder 30 may be further equipped with a fixture 89, e.g. in form of a single or numerous grooves on the inside of the frame 30 one. The fixture 89 is shaped and adapted to receive at least a portion of an infusion line 88. An inside cross section of a groove of the fixture 89 may be complementary-shaped to the diameter of the infusion line 88, such that the flexible infusion line 88 can be clamped into the fixture 89, thereby fixing or stabilizing the infusion line 88 to the holder 30.

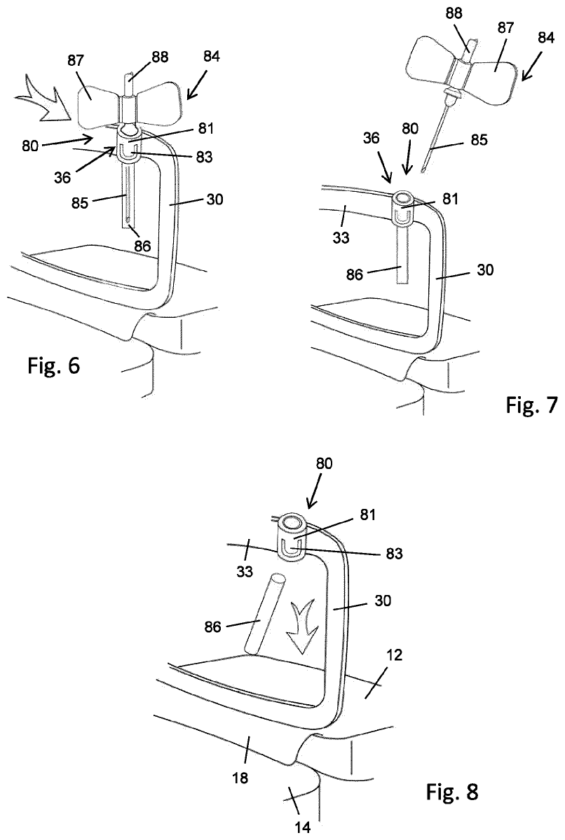

The holder 30 is further provided with another mount 36 implemented as a needle unit holder 80, the structure and functionality of which being explained in greater detail by the examples as shown in FIG. 6-12. The needle unit holder 80 comprises a sleeve 81 with a tubular-shaped sidewall 82. The sidewall 82 may be of cylindrical or conical shape. The sleeve 81 is configured to receive at least a portion of a needle unit 84, which needle unit 84 is provided with a needle 85 and a needle cap 86 covering the needle 85. The needle cap 86 is detachably connected to the needle unit 84. The needle unit 84 may further comprise a handle 87, e.g. implemented as butterfly flaps.

A user may insert the needle unit 84 along a first direction into the sleeve 81 of the needle unit holder 80 until a stop configuration of final assembly configuration has been reached. In this configuration the needle 86 is fastened and fixed inside the sleeve 81. When the user then retracts the needle unit 84, e.g. by applying a lifting force or a respective pull force to the needle unit 84, e.g. via the handle 87, the needle cap 86 automatically releases from the needle unit 84 and the needle unit 84 with the needle 85 may leave the needle cap 86 as e.g. shown in FIGS. 7 and 11. After removal of the needle 85 and the needle unit 84 from the needle cap 86 the needle cap 86 may be easily removed from the needle holder 80 or sleeve 81, by pulling the needle cap 86 in the same direction as it has been initially inserted into the sleeve 81.

The needle cap 86 may be fastened inside the sleeve 81 by friction and/or by a form fit as it is e.g. apparent from FIGS. 14 and 15. In some examples the sleeve 81 may comprise a radially inwardly extending fastening flap 83 provided with a distally facing stop face 44. When reaching a final assembly configuration as illustrated in FIG. 15 a complementary shaped proximally facing counter stop face 45 as provided on the outside of the needle cap 86 may get in longitudinal engagement with the stop face 44.

Accordingly, the mutual engagement of the stop face 44 with the counter stop face 45 prevents an upwardly and hence proximally directed movement of the needle cap 86 relative to the sleeve 81. Once a final assembly configuration as illustrated in FIG. 15 has been reached the needle 86 cap cannot be taken out of the sleeve 81 in a direction opposite to the direction of insertion.

The same or similar effect may apply when the outside surface of the needle 86 should be void of a stop face or recess as shown in FIG. 14. Here, a respective friction force between the radially inwardly extending fastening slap or fastening flaps 83 and an outside surface of the needle cap 86 is sufficient to prevent a respective movement in the upward or second direction.

The fastening flap 83 may be integrally formed with the sidewall 82 of the sleeve 81. It may be provided by a U-shaped through opening in the sidewall 82 and may be biased radially inwardly to exert a respective radially inwardly extending holding or fixing force onto the needle cap 86.

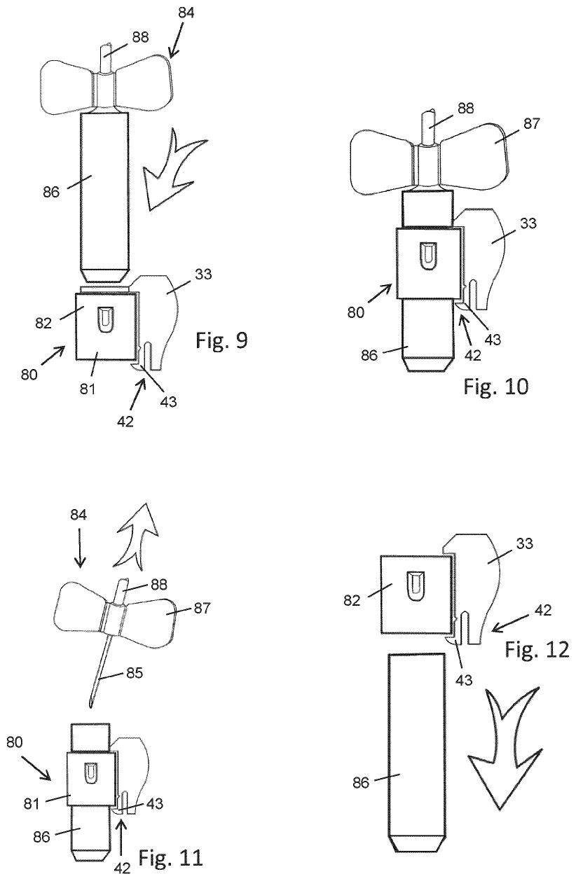

With the example according to FIGS. 9-13 the needle unit holder 80 is provided as a separate piece or as a separate part detachably connected to the holder 30 or traverse 33. Here, the traverse 33 may be provided with a fastener 42, e.g. implemented as a snap fastener 43 by way of which the sleeve 81 and hence the needle unit holder 80 can be fastened or connected to the holder 30. The needle unit holder 80 and/or its sleeve 81 may be provided or implemented as a metal part, whereas the holder 30 may be formed of a plastic material. The clip-type fastening as provided by the fastener 43 allows for a detachable connection of the needle unit holder 80 with the traverse 33.

The needle unit holder 80 and the separation of the needle cap 86 from the needle unit 84 is beneficial for a single-handed handling of the needle unit 84 in the course of preparing an injection or infusion procedure. Thus, the needle unit holder 80 in the elevated or raised configuration 80 is of particular benefit for a one-handed operation of the needle unit 84 in the course of preparing or executing at least one of an injection procedure and an infusion procedure.

The holder 30 is further provided with another mount 34, which is implemented to hold at least one of an illumination device 6 and an optical device 7. The illumination device 6 is further illustrated in FIGS. 17 and 19. The illumination device 6 comprises a plug 71 and the mount 34 comprises a complementary shaped socket 70. The illumination device 6 can be mechanically connected to the holder 30 by inserting the plug 71 into the socket 70. The illumination device 6 may comprise a gooseneck or gooseneck portion 72 by way of which a light source 73 of the illumination device 6 is mechanically connected to the plug 71. The plug 71 is mechanically connectable to the socket 70 and hence to the holder 30. The gooseneck portion 72 allows for a rather flexible orientation or position of the light source 73. The light source 73 or any other part of the illumination device 6 may be further equipped with a switch 74 or with a respective actuation or control element by way of which the light can be switched on or switched off.

There may be also provided on optical device 7, e.g. implemented as a magnifier comprising a magnifying glass 73′ e.g. connected via a gooseneck portion 72′ with a plug 71′. As described above in connection with the illumination device 6, also the optical devices 7 may be detachably connected with e.g. the mount 34 a with another mount (not illustrated). The magnifying glass 73′ may be oriented in a rather flexible and variable manner relative to the plug 71′ thus allowing to move the magnifying glass 73′ in a desired position and/or orientation relative to the holder 30 and hence relative to the armrest 50 in order to optically magnify a portion of a patient's skin that should be subject to injection or infusion.

In some examples the illumination device 6 is provided with an energy supply, such as a battery. It may then operate stand alone and irrespective of its connection with the older 30. With other examples the socket 70 may be electrically connected to a battery 95 or power source of the patient care kit thus allowing to provide an electric power supply for the light source 73 when appropriately attached to the mount 34.

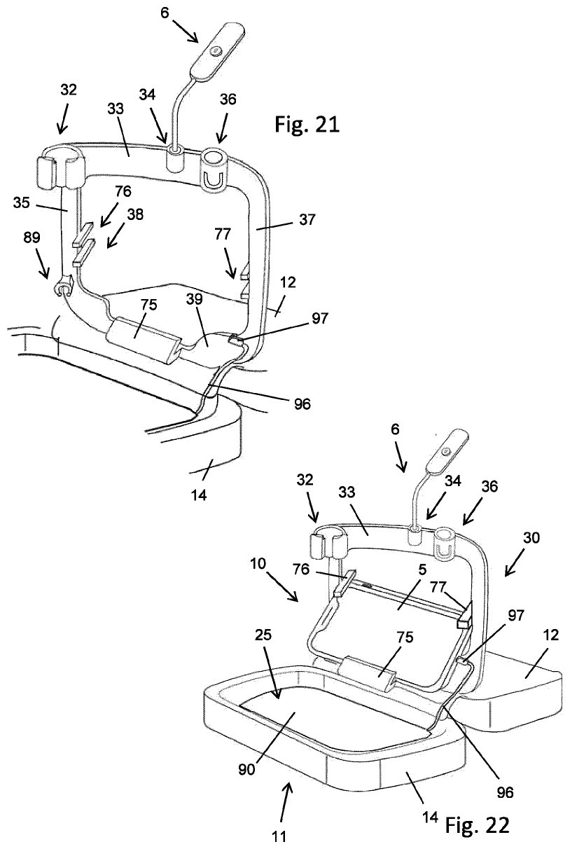

The frame 30 is further equipped with a mount 38 implemented as a device mount for a portable electronic device 5, e.g. implemented as a smart phone. The mount 38 is particularly illustrated in FIGS. 21 and 22. The mount 38 comprises an abutment 75, e.g. located on or in the bottom traverse 39 of the holder 30. The mount 38 further comprises a first guiding structure 76 on an inside of the first leg 35 and further comprises another, hence, a second guiding structure 77 on an inside of the second leg 37. The guiding structures 76, 77 allow for a sliding inserting of the portable electronic device 5 into the mount 38 until an edge of the housing of the portable electronic device 5 engages with the abutment 75. The guiding structures 76, 77 may be arranged in an inclined or tilted manner with respect to a planar structure of the frame 31 of the holder 30. In this way and with an upright oriented holder 30 as illustrated in FIG. 22 there can be provided a well-defined inclined or tilted orientation of the portable electronic device 5 in the mount 38. This allows for a good and intuitive visibility and readability of a display of the portable electronic device 5 when arranged or attached to the mount 38.

In FIG. 21 there is further illustrated another fixture 89 for fixing the infusion line 88 to the holder 30. This fixture 89 is implemented as a clip protruding from a plane of the holder 30.

As illustrated in the block diagram of FIG. 19 the patient care kit 10 may be provided with an illumination module 90, the details of which are further apparent from FIG. 20. The illumination module 20 may be arranged on a bottom of the receptacle 25 of the second housing component 30. The illumination module comprises a surface 91 facing upwardly. The surface 91 is provided with numerous light sources 92, 93, e.g. implemented as LED light sources. The individual light sources 92, 93 may be independently activated or deactivated, e.g. by an illumination controller. With the presently illustrated example the illumination module 90 may be void of an own controller. Here, a respective illumination controller may be provided by the external electronic device 5. For this, the illumination module 90 is connectable with the external electronic device in a signal transferring manner.

As shown in FIGS. 19-22 there may be provided a transmission line 96, e.g. provided with plugs 97, 97′ at opposite ends. The plug 97 may be connected with a socket 98 of the external electronic device 5. The plug 97′ may be connected with a complementary shaped socket 98′ as provided on or in the housing 11 of the patient care kit 10. The socket 98′ is electrically connected to the illumination module 90. Alternatively, the transmission line 96 may be directly connected or integrated into the illumination module 90.

Optionally, the illumination module 90 and/or the patient care kit 10 is provided with a communication interface 94 operable to wirelessly communicate with a corresponding communication interface 94′ of the external electronic device 5. This way, control signals as generated by an app or software routine executed by the external electronic device 5 can be transmitted to the illumination module to selectively activate and/or to selectively deactivate individual light sources 92, 93. Optionally, and as shown in FIG. 19 the patient care kit 10 may be provided with an own battery 95. The battery 95 may be also connected to the socket 98′ or to another socket 98″. The socket 98″ may be configured to connect to a plug 97″ of an external or further battery 95′. The external battery 95′ may be also replaced by a stationary power source.

The battery 95 may be implemented as a rechargeable battery, which can be charged by the external battery 95′, by an external power source and/or by the battery 95″ of the external electronic device 5, when connected to the battery 95 through the transmission line 96 or through a wireless power transferring connection.

Optionally, the mount 34 configured to hold the illumination device 6 may be also electrically coupled to the battery 95 or to any of the sockets 98′, 98″. The illumination device 6 may comprise an own battery or power source and may be operated standalone. However, the plug 71 of the illumination device 6 may comprise an integrated electrical contact 69 to make electrical contact with a corresponding counter contact 69′ of the socket 70. This way and when the illumination device 6 is connected via its plug 71 or connector with the socket 70 or counter-connector of the mount 44, the light source 73 may be either provided with electrical power from the battery 95 or from the battery 95″ of the external electronic device 5, when the external electronic device 5 is connected or coupled to the patient care kit 10 in an electric power transmitting manner. Additionally, a local battery as provided in or on the illumination device 6 may be charged or recharged by any of the batteries 95, 95′, 95″.

In some examples the patient care kit 10 is void of an own battery 95. It may receive electrical power exclusively through a power transmitting coupling with the external electronic device 5.

In a scenario of use and after having transferred the housing 11 of the patient care kit 10 into the configuration of use the external electronic device 5 may be mounted to the mount 38 in a tilted or inclined manner as indicated in FIG. 22. The external electronic device 5 may be further connected with the illumination device 90, e.g. via the transmission line 96. For this, the plug 97 has to be manually inserted into the socket 98. Alternatively, the communication interface 94 of the patient care kit 10 couples wirelessly with the corresponding communication interface 94′ of the external electronic device 5.

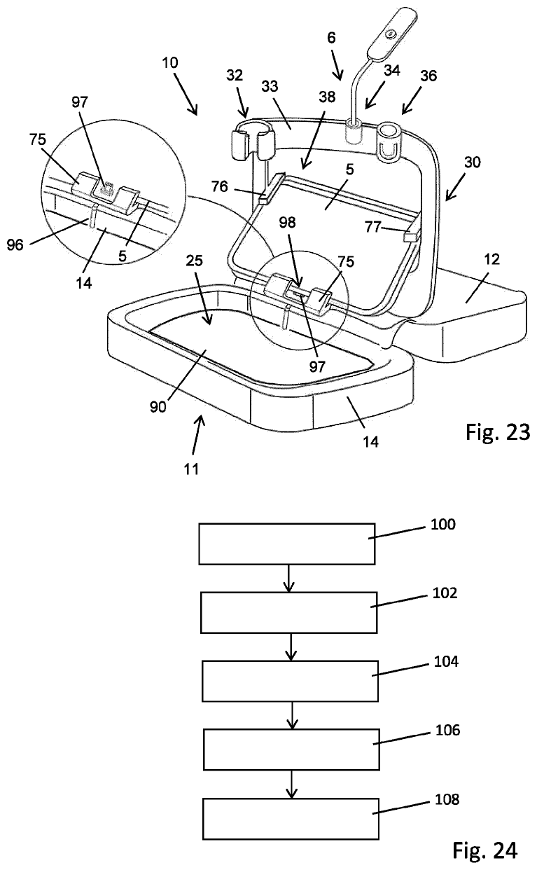

In a further example as shown in FIG. 23, the mount 38 for the external electronic device 5 may comprise the plug 97, which is complementary shaped to the socket 98 of the external electronic device 5. Hence, the plug 97 may be integrated in one of the mount 38, e.g. in one of the bottom traverse 39 and the abutment 75. Here, the plug 97 may be fixed to or relative to the mount 38 and/or relative to the guiding structures 76, 77. The plug 97 may be positioned and arranged on or inside the mount 38 to automatically connect to the socket 98 of the external electronic device 5 when the external electronic device 5 reaches a pre-defined assembly or fastening position in or with the mount 38.

Specifically, the guiding structures 76, 77 and the plug 97 may be arranged and aligned relative to each other such that a plug-socket connection is obtained at the end of an e.g. sliding insert motion of the electronic device 5 into or relative to the mount 38. Hence, the plug 97 is positioned on or in the mount 38 in such a way that by way of a guiding of the external electronic device 5 as provided by the guiding structures 76, 77, the plug 97 is correctly aligned with the socket 98. A mutual mechanical and/or electrical connection between the plug 97 and the socket 98 may then define an end-position or final assembly position of the external electronic device 5 relative to the mount 38. Specifically, the plug 97 may be arranged on, in or inside the abutment 75 and may be oriented and/or aligned along a sliding or insert direction as defined by guiding structures 76, 77.

The illumination device 90 with its individual light sources 92, 93 is typically covered by the inlay 22, which is typically equipped with numerous items 8. A user may initialize a training or guiding app on the external electronic device 5, which when executed by a processor of the electronic device 5 may visually indicate numerous steps of using the patient care kit 10 and/or for preparing a medicament and/or for preparing or executing at least one of an injection procedure and an infusion procedure. The external electronic device 5 may be configured to visually indicate numerous steps of use on a display.

Here, the user may be prompted e.g. to take a particular item 8 out of a first compartment 20. Correspondingly to the user instruction as indicated by the electronic device 5, the electronic device 5 may also activate a light source 92 located underneath a compartment 20, in which the item 8 to be gripped by a user is currently located. Subsequently and when the user may e.g. have prompted to have removed the respective item 8 from the compartment 20 the instructions as provided by the electronic device 5 may continue to prompt the user to remove another item from another compartment 21, which overlaps or spatially coincides with another light source 93. Concurrent with an indication to the user to remove the further item 8 from the further compartment 21 the electronic device 5 may deactivate the first light source 92 and may activate the second light source 93, thereby guiding the user to take or to remove the respective item from the further compartment 21. This way guiding or assisting a user in using the patient care kit 10 and the numerous items 8 stored therein becomes rather intuitive and straightforward to the user.

The flowchart of FIG. 24 is indicative of a method of using the portable patient care kit 10 as described herein. In a first step 100 the housing 11 of the portable patient care kit 10 may be transferred from a closed configuration into the configuration of use. For this, a user may release a connection between the first and the second housing components 12, 14 and may pivot at least one of the first and the second housing components 12, 14 relative to the other one of the first and the second housing components 12, 14.

Thereafter and in step 102 the user may install or move the holder 30 into the protruding or upright configuration as e.g. illustrated in FIG. 1. Thereafter and in step 104 the user may install the electronic device 5 in the prescribed manner to the patient care kit 10. For instance, the external electronic device 5 may be mounted in the mount 38 as provided by the holder 30. In step 106 the electronic device 5 may be coupled with the illumination module 90, e.g. by inserting the plug 97 into the socket 98.

In step 108 a user may start an app on the electronic device 5 and may then start to follow the instructions as provided by the electronic device 5. By way of these instructions the user may then e.g. prompted to remove a particular item, e.g. a medicament container 8 out of a compartment 20. The user may be further prompted to remove an injection device 1 out of a compartment 21 and to mount the injection device 1 to the mount 32.

In a further step the user may be prompted to connect the medical device 1 with a selected medical device accessory 2. Hence, a user may be prompted to attach an infusion line 88 to an outlet 68 of a syringe and to position or to fix the syringe 1 and the infusion line 88 to the mount 30 as e.g. illustrated in FIG. 18.

Thereafter, the user may be prompted to remove a needle unit 84 from a respective compartment 20 or 21 and to mount the needle unit 84 to a needle unit holder 80 as described in the sequence of FIGS. 6-15. Here, and after having mounted the needle unit 84 to the mount 36 or needle unit holder 80 the user may withdraw the needle unit 84 from the needle unit holder 80, thereby inherently disconnecting a protective cap or needle cap 86 from the needle 85.

With all these individual steps, the portable patient care kit 10 with its holder 90 is of particular benefit to assist or to guide a user in preparing of a medicament and/or to assist or to guide the user in preparing at least one of an infusion procedure or injection procedure.

Furthermore, the portable patient care kit 10 provides a safe and reliable housing 11 for storing and/or transporting numerous items 8 for self-medication or home-medication.

### REFERENCE NUMBERS

1 medical device
2 medical device accessory
3 medicament container
4 dressing material
5 portable electronic device
6 illumination device
7 optical device
8 item
9 support
10 patient care kit
11 housing
12 housing component
13 inside
14 housing component
15 inside
16 outside
17 outside
18 intermediate section
19 inside
20 compartment
21 compartment
22 inlay
24 housing pivot axis
25 receptacle
26 sidewall
27 sidewall
28 sidewall
29 sidewall
30 holder
31 frame
32 mount
33 traverse
33a first end
33b second end
34 mount
35 leg
35a first end
35b second end
36 mount
37 leg
37a first end
37b second end
38 mount
39 bottom traverse
40 hinge
41 hinge
42 fastener
43 snap fastener
44 stop face
45 counter stop face
46 fastener
47 counter fastener
48 zipper
50 armrest
51 body
52 base portion
53 extension
54 extension
55 pivot axis
56 pivot axis
57 flap
58 flap axis
59 pocket
60 slot
61 sidewall
62 opening
63 protrusion
64 barrel
65 plunger
66 finger flange
68 outlet
69 contact
70 socket
71 plug
72 gooseneck portion
73 light source
74 switch
75 abutment
76 guiding structure
77 guiding structure
80 needle unit holder
81 sleeve
82 sidewall
83 fastening flap
84 needle unit
85 needle
86 needle cap
87 handle
88 infusion line
89 fixture
90 illumination module
91 surface
92 light source
93 light source
94 communication interface
95 battery
96 transmission line
97 plug
98 socket

1-18. (canceled)

19. A portable patient care kit comprising:
a housing comprising a first housing component and a second housing component, wherein the second housing component is movably connected to the first housing component and wherein the housing is reconfigurable between a closed configuration and a configuration of use by moving the second housing component relative to the first housing component,
at least one compartment provided on an inside of one of the first housing component and the second housing component and configured to accommodate an item selected from at least one of a medical device, a medical device accessory, a medicament container, dressing material, a portable electronic device, an illumination device, and an optical device,
a holder comprising a mount for fastening the item or an external item (8′) to the holder and wherein the holder is connected or is connectable to at least one of the first housing component and the second housing component to protrude from the inside of one of the first housing component and the second housing component when the housing is in the configuration of use.

20. The patient care kit according to claim 19, wherein the holder is pivotably attached to at least one of the inside of first housing component, the inside of the second housing component and an inside of an intermediate section located between the first housing component and the second housing component via a hinge.

21. The patient care kit according to claim 19, wherein the holder comprises a first leg and a second leg and a longitudinally extending end, wherein the first leg is connected to a first longitudinal end of the end and wherein the second leg is connected to a second longitudinal end of the end and wherein the mount is provided on or in the end.

22. The patient care kit according to claim 21, wherein the holder comprises a closed frame.

23. The patient care kit according to claim 19, wherein when the housing is in the configuration of use, the holder is in an upright position or is configured to reach an upright position, in which the mount is in an elevated position relative to the inside of at least one of the first housing component and the second housing component.

24. The patient care kit according to claim 19, further comprising an armrest connectable or connected with the inside of one of the first housing component and the second housing component.

25. The patient care kit according to claim 24, wherein the armrest comprises a body with a base portion connected or connectable with one of the first housing component and the second housing component and at least one armrest extension pivotally connected to the base portion and pivotable relative to the base portion between an extended configuration and a storage configuration, wherein when in the storage configuration the armrest extension overlaps with the base portion and wherein when in the extended configuration the armrest extension extends beyond at least one of the first housing component and the second housing component.

26. The patient care kit according to claim 19, wherein the mount is configured to detachably hold at least one of an injection device and a medicament container in an upright orientation.

27. The patient care kit according to claim 19, wherein the mount is configured hold and/or to fix at least one of an illumination device and an optical device.

28. The patient care kit according to claim 27, wherein the mount comprises a socket to receive a complementary shaped plug of the illumination device or optical device.

29. The patient care kit according to claim 19, wherein the mount comprises a needle unit holder configured to detachably hold and/or fix a needle unit.

30. The patient care kit according to claim 19, wherein the mount is configured to removably receive a portable electronic device.

31. The patient care kit according to claim 19, wherein at least one of the first and the second housing component and the second housing component comprises a receptacle to receive an inlay comprising the at least one compartment.

32. The patient care kit according to claim 19, further comprising an illumination module arranged inside at least one of the first housing component and the second housing component and comprising a number of individual light sources distributed across a surface of the illumination module and overlapping with a position of the at least one compartment

33. The patient care kit according to claim 32, wherein the illumination module comprises a communication interface to communicate with an external electronic device operable to selectively control operation of the individual light sources of the illumination module.

34. A method of preparing a medicament and/or of preparing administering a medicament to a patient by using a portable patient care kit according to any one of the preceding claims, the method comprising at least one or several of the following steps:
transferring the housing of the portable patient care kit from a closed configuration into the configuration of use by moving the first housing component relative to the second housing component,
transferring or moving of the holder of the portable patient care kit into the protruding or raised configuration,
removing of the at least one item stored in or provided in the compartment of the portable patient care kit and positioning and/or fixing the item to the mount of the holder,
preparing a medicament and/or preparing at least one of an injection procedure or infusion procedure with the item positioned in an elevated or raised position relative to at least one of the first housing component and the second housing component.

35. The method according to claim 34, further comprising the steps of:
attaching or mounting an external electronic device to a device mount of the holder,
starting of a software on the external electronic device and assisting or guiding a user executing at least one of preparing of the medicament and/or preparing at least one of the injection or infusion.

36. The method according to claim 34, further comprising the steps of:
electronically coupling an external electronic device with an illumination module of the portable patient care kit, the illumination module comprising a first light source and a second light source, wherein the first light source overlaps with a first compartment and wherein the second light source overlaps with the second compartment provided on the inside of at least one of the first housing component and the second housing component,
separately and/or consecutively illuminating the first and the second compartments by the first light source and by the second light source of the illumination module, respectively.

37. The method according to claim 35, further comprising the steps of:
electronically coupling an external electronic device with an illumination module of the portable patient care kit, the illumination module comprising a first light source and a second light source, wherein the first light source overlaps with a first compartment and wherein the second light source overlaps with the second compartment provided on the inside of at least one of the first housing component and the second housing component,
separately and/or consecutively illuminating the first and the second compartments by the first light source and by the second light source of the illumination module, respectively.
